(19) United States
(12) Patent Application Publication
(10) Pub. No.: US 2026/0022407 A1
(43) Pub. Date: Jan. 22, 2026

(54) METHOD, ENZYME COMPOSITION, NUCLEIC ACID COMPOSITION, AND TRANSGENIC MICROORGANISM FOR PRODUCING ISOPRENE GLYCOL

(71) Applicant: National Yang Ming Chiao Tung University, Hsinchu City (TW)

(72) Inventors: Ethan I. LAN, Hsinchu City (TW); You-An CHEN, Hsinchu County (TW); Jian-Dong LAI, Taipei City (TW)

(21) Appl. No.: 18/988,809

(22) Filed: Dec. 19, 2024

(30) Foreign Application Priority Data
Jul. 22, 2024 (TW) 113127377

(51) Int. Cl. C12P 7/18; C12N 9/02; C12N 9/10; C12N 9/16; C12N 9/88; C12N 15/52

(52) U.S. Cl. CPC C12P 7/18; C12N 9/0008; C12N 9/1025; C12N 9/1029; C12N 9/16; C12N 9/88; C12N 15/52; C12Y 102/99006; C12Y 203/01009; C12Y 203/0301; C12Y 301/0202; C12Y 402/01018

(57) ABSTRACT

A method for producing isoprene glycol, including: enzymatically generating 3-hydroxy-3-methylbutyryl-CoA (HMB-CoA) from acetyl-CoA (Ac-CoA); enzymatically generating 3-methyl-3-hydroxybutyrylaldehyde (3-HMBA) from HMB-CoA; and enzymatically converting 3-HMBA to isoprene glycol (ISPG). Wherein the enzymatically generating 3-HMBA from HMB-CoA includes: enzymatically reducing HMB-CoA to produce 3-HMB; or enzymatically hydrolyzing HMB-CoA to 3-hydroxy-3-methylbutyric acid (HMB) and then enzymatically reducing HMB to 3-HMBA. Also provided herein are an enzyme composition, a nucleic acid composition, and a transgenic microorganism for producing isoprene glycol.

### CROSS-REFERENCE TO RELATED APPLICATION

This application claims priority to Taiwan Application Serial Number 113127377, filed Jul. 22, 2024, which is herein incorporated by reference in its entirety.

The Sequence Listing associated with this application is filed in electronic format via EFS-Web and is hereby incorporated by reference into the specification in its entirety. The name of the XML file containing the Sequence Listing is NP-36222-US_SEQ_LIST.xml. The size of the XML file is 21,553 bytes, and the XML file was created on Nov. 6, 2024.

### BACKGROUND

### Field of Invention

The present disclosure relates to transgenic microorganisms, enzyme compositions, nucleic acid compositions, and methods for producing isoprene glycol through biosynthesis.

### Description of Related Art

Isoprene glycol (ISPG) is a colorless, odorless, highly water-soluble organic solvent that is widely used as an emulsifier, moisturizer, and hair conditioner in personal care and cosmetic products, and it is biodegradable.

Currently, the primary method for producing isoprene glycol is through chemical synthesis. A typical reaction involves using formaldehyde and isobutylene as raw materials in a Prins reaction to form isopentenol, which is then hydrated in a phosphoric acid solution to synthesize isoprene glycol. This method was initially developed for the production of isoprene. However, this method has several limitations. The reaction conditions for this method are more stringent, requiring a high-temperature and high-pressure environment throughout the process. This necessitates the use of more advanced equipment and technical specifications. The catalysts used, such as phosphoric acid or sulfuric acid, are corrosive. Additionally, there is a dependency on petrochemical raw materials. Therefore, more research teams are looking to find green and environmentally friendly bioprocesses to replace the traditional chemical synthesis.

Accordingly, finding a biosynthetic pathway to produce isoprene glycol is a pressing issue to be addressed.

### SUMMARY

In light of the aforementioned issues, embodiments of the present disclosure present a biosynthetic pathway for producing isoprene glycol. This pathway involves the generation of HMB-CoA from acetyl-CoA, which serves as a precursor, followed by the conversion of HMB-CoA into isoprene glycol. Since acetyl-CoA can be derived from glycolysis, glucose can serve as the carbon source. As glucose is a readily renewable resource, this approach for producing isoprene glycol is environmentally friendly.

Some embodiments of the present disclosure provide a method for producing isoprene glycol, including: enzymatically generating 3-hydroxy-3-methylbutyryl-CoA (HMB-CoA) from acetyl-CoA (Ac-CoA); enzymatically generating 3-methyl-3-hydroxybutyrylaldehyde (3-HMBA) from HMB-CoA; and enzymatically converting 3-HMBA to isoprene glycol (ISPG). The enzymatically generating 3-HMBA from HMB-CoA includes: enzymatically reducing HMB-CoA to 3-HMBA; or enzymatically hydrolyzing HMB-CoA to 3-hydroxy-3-methylbutyric acid (HMB) and then enzymatically reducing HMB to 3-HMBA.

In some embodiments, enzymatically generating HMB-CoA from acetyl-CoA includes: enzymatically converting acetyl-CoA to acetoacetyl-CoA (AcAc-CoA); enzymatically converting AcAc-CoA to 3-hydroxy-3-methylglutaryl coenzyme A (HMG-CoA); enzymatically converting HMG-CoA to 3-methylglutaconyl-CoA (3-MG-CoA); enzymatically converting 3-MG-CoA to 3-methyl crotonyl-CoA (3-MC-CoA); and enzymatically converting 3-MC-CoA to 3-hydroxy-3-methylbutyryl-CoA (HMB-CoA).

Some embodiments of the present disclosure provide an enzyme composition for producing isoprene glycol, including: a first group, and a second group or a third group. The first group includes: acetyl-CoA acetyltransferase (AtoB), 3-hydroxy-3-methylglutaryl coenzyme A (HMG-CoA) synthase (MvaS), HMG-CoA dehydratase/methylcrotonyl-CoA (MC-CoA) hydratase (LiuC), and methylglutaconyl-CoA (MG-CoA) decarboxylase (AibA/AibB). The second group includes coenzyme A-acylating aldehyde dehydrogenase. The third group includes thioesterase (YciA) and carboxylic acid reductase (CAR).

In some embodiments, the enzyme composition for producing isoprene glycol further includes pantothenate kinase, 4′-phosphopantetheinyl transferase, or aldehyde reductase.

Some embodiments of the present disclosure provide a nucleic acid composition for producing isoprene glycol, including: at least one gene vector configured to express the enzyme composition for producing isoprene glycol as described in the embodiments above or below. For example, the first group of genes encodes: AtoB, MvaS, LiuC, and AibA/AibB. The second group of genes encodes coenzyme A-acylating aldehyde dehydrogenase. The third group of genes encodes thioesterase (YciA) and carboxylic acid reductase (CAR). In some embodiments, at least one gene vector further encodes pantothenate kinase, 4′-phosphopantetheinyl transferase, or aldehyde reductase.

Some embodiments of the present disclosure provide a transgenic microorganism for producing isoprene glycol, including: an acetyl-CoA generation pathway that produces acetyl-CoA from a carbon source; a first stage pathway that generates 3-hydroxy-3-methylbutyryl-CoA (HMB-CoA) from acetyl-CoA; a second (A) stage pathway or a second (B) stage pathway, wherein the second (A) stage pathway reduces HMB-CoA to produce 3-methyl-3-hydroxybutyrylaldehyde (3-HMBA), and the second (B) stage pathway hydrolyzes HMB-CoA to 3-hydroxy-3-methylbutyric acid (HMB) and then reduces HMB to 3-HMBA; and a third-stage pathway that reduces 3-HMBA to isoprene glycol (ISPG).

In some embodiments, the transgenic microorganism for producing isoprene glycol includes: a host cell, a first set of heterologous genes, and a second set of heterologous genes or a third set of heterologous genes. The first set of heterologous genes, the second set of heterologous genes, and the third set of heterologous genes are located within the host cell. The genome of the host cell encodes genes for the acetyl-CoA generation pathway. The first set of heterologous genes is configured to express the enzymes of the first stage pathway. The second set of heterologous genes is configured to express the enzymes of the second (A) stage pathway, while the third set of heterologous genes is configured to express the enzymes of the second (B) stage pathway.

In some embodiments, the genome of the host cell also encodes an endogenous aldehyde reductase for the third stage pathway.

In some embodiments, the enzymes of the first stage pathway include acetyl-CoA acetyltransferase (AtoB), 3-Hydroxy-3-methylglutaryl coenzyme A (HMG-CoA) synthase (MvaS), HMG-CoA dehydratase/methylcrotonyl-CoA (MC-CoA) hydratase (LiuC), and methylglutaconyl-CoA (MG-CoA) decarboxylase (AibA/AibB).

In some embodiments, the AtoB has at least 80% identity to SEQ ID NO: 1. The MvaS has at least 70% identity to SEQ ID NO: 2. The LiuC has at least 70% identity to SEQ ID NO: 3. The methylglutaconyl-CoA (MG-CoA) decarboxylase includes MG-CoA decarboxylase subunit A (AibA) and MG-CoA decarboxylase subunit B (AibB), wherein the AibA has at least 70% identity to SEQ ID NO: 4, and the AibB has at least 70% identity to SEQ ID NO: 5.

In some embodiments, the first set of heterologous genes further encodes pantothenate kinase configured to supplement coenzyme A (CoA).

In some embodiments, the pantothenate kinase has at least 60% identity to SEQ ID NO: 10.

In some embodiments, the second set of heterologous genes includes coenzyme A-acylating aldehyde dehydrogenase.

In some embodiments, the coenzyme A-acylating aldehyde dehydrogenase has at least 60% identity to SEQ ID NO: 7, SEQ ID NO: 12, or SEQ ID NO: 13.

In some embodiments, the third set of heterologous genes encodes thioesterase (YciA) and carboxylic acid reductase (CAR).

In some embodiments, the YciA has at least 60% identity to SEQ ID NO: 6, and the CAR has at least 60% identity to SEQ ID NO: 8, SEQ ID NO: 14, or SEQ ID NO: 15.

In some embodiments, the third set of heterologous genes further encodes 4′-phosphopantetheinyl transferase to activate carboxylic acid reductase (CAR).

In some embodiments, the 4′-phosphopantetheinyl transferase has at least 60% identity to SEQ ID NO: 9.

In some embodiments, the transgenic microorganism for producing isoprene glycol also includes: a fourth set of heterologous genes encoding an heterologous aldehyde reductase configured to facilitate the third stage pathway.

In some embodiments, the heterologous aldehyde reductase has at least 60% identity to SEQ ID NO: 11.

In some embodiments, the first set of heterologous genes, and the second set or third set of heterologous genes are located on a same plasmid.

In some embodiments, the first set of heterologous genes, and the second set or third set of heterologous genes are located on two or more plasmids.

Some embodiments of the present disclosure also provide a method for producing isoprene glycol, including: providing a transgenic microorganism as described in the embodiments above or below; and cultivating the transgenic microorganism in a culture medium, wherein the carbon source in the culture medium includes glucose.

In some embodiments, the method for producing isoprene glycol further includes adding vitamin B5 to the culture medium.

### BRIEF DESCRIPTION OF THE DRAWINGS

The invention can be more fully understood by reading the following detailed description of the embodiment, with reference made to the accompanying drawings as follows:

FIG. 1A illustrates the biosynthetic pathway of isoprene glycol according to some embodiments of the present disclosure.

FIGS. 1B and 1C show the structures of the compounds involved in the biosynthetic pathway of isoprene glycol as depicted in FIG. 1A.

FIG. 2A illustrates the biosynthetic pathway of isoprene glycol and the associated enzymes in a transgenic microorganism.

FIG. 2B illustrates the biosynthetic pathway of isoprene glycol and the associated enzymes in a transgenic microorganism.

FIG. 3A illustrates a plasmid designed according to some embodiments.

FIG. 3B shows the test results of the plasmid in FIG. 3A.

FIG. 4A illustrates a plasmid designed according to some embodiments.

FIG. 4B shows the test results of the plasmid in FIG. 4A.

FIG. 5A illustrates a plasmid designed according to some embodiments.

FIG. 5B shows the test results of the plasmid in FIG. 5A.

FIG. 5C illustrates the phylogenetic tree of the CAR enzymes in FIGS. 5A and 5B, based on the sequences of the CAR enzymes.

FIG. 6A illustrates a plasmid designed according to some embodiments.

FIG. 6B shows the test results of the plasmid in FIG. 6A.

FIG. 7A illustrates the plasmid designed according to some embodiments.

FIG. 7B shows the test results of the plasmid in FIG. 7A.

FIG. 7C shows the test results of the plasmid in FIG. 7A.

FIG. 8A illustrates a schematic of the metabolic pathway from glucose to isoprene glycol and the reactions in which coenzyme A is involved in this pathway.

FIG. 8B shows the test results of expressing the enzyme CoaA, which enhances coenzyme A, in the transgenic microorganisms, without the addition of vitamin B5 during cultivation.

FIG. 8C shows the test results of expressing the enzyme CoaA, which enhances coenzyme A, in the transgenic microorganisms, with the addition of vitamin B5 during cultivation.

FIG. 9A shows the concentrations of isoprene glycol in the culture medium after the transgenic microorganism were cultured for different days.

FIG. 9B shows the concentrations of glucose and the growth rates of the microorganism in the culture medium after the transgenic microorganisms were cultured for different days.

FIG. 9C shows the concentrations of various metabolites in the culture medium after the transgenic microorganisms were cultured for different days.

### DETAILED DESCRIPTION

The following will clearly explain the spirit of the present disclosure with detailed descriptions and figures. It should be understood that the content of the present disclosure can have various changes in different forms, but all of them remain within the scope of the present disclosure. The explanations and accompanying figures are intended for clarification purposes and are not meant to limit the content of the present disclosure.

In various embodiments of the present disclosure, a pathway has been constructed to produce isoprene glycol using derivatives of glucose metabolism. Since glucose is a renewable organic carbon source that can be utilized by most microorganisms, this approach can reduce dependence on petrochemical raw materials and decrease harmful byproducts.

As used herein, the term “endogenous” refers to a nucleic acid sequence that naturally exists within a microorganism.

As used herein, the term “heterologous” refers to substances that are not naturally present in a host cell. For example, a nucleic acid sequence is considered heterologous to the host cell if at least one of the following conditions is met: (a) the nucleic acid is not naturally found in that cell (meaning it is “exogenous” nucleic acid); (b) the nucleic acid is naturally found in the given host cell (meaning it is “endogenous”), but the nucleic acid or the RNA or protein produced by the transcription and translation of this nucleic acid is present in non-natural (for example, greater or lesser than naturally occurring) amounts in the host cell; (c) the nucleic acid encodes an endogenous protein of the host cell, but the nucleotide sequence differs in terms of sequence from the endogenous nucleotide sequence that encodes the same protein (having the same or substantially similar amino acid sequence), which generally leads to the protein being produced in larger amounts in the cell; or in the case of enzymes, produces mutated forms with altered (for example, higher or lower or different) activity; and/or (d) the nucleic acid contains two or more nucleotide sequences that are not found in the same relationship to each other in the host cell.

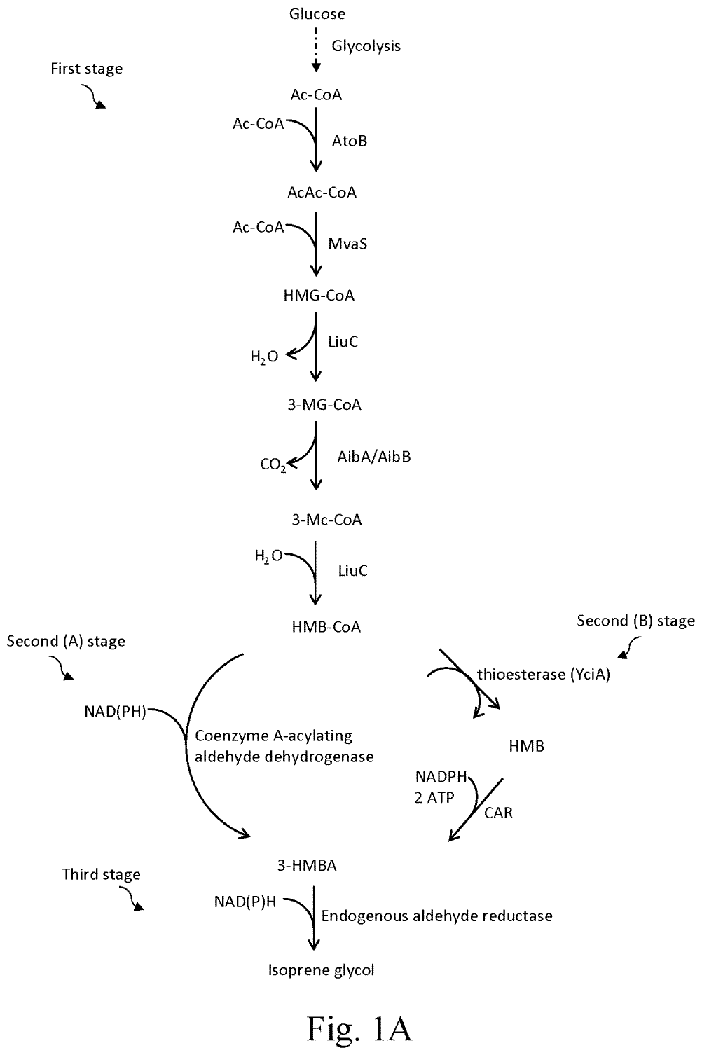

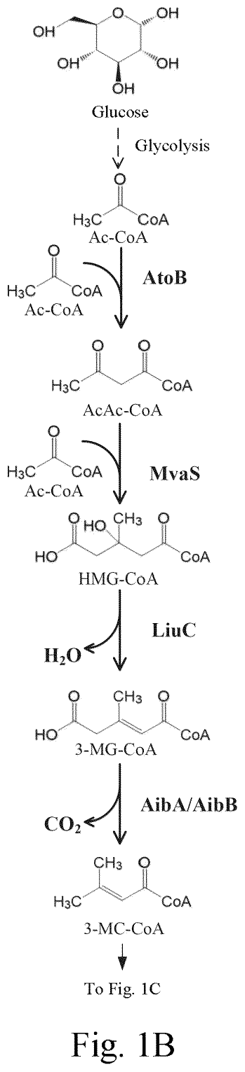

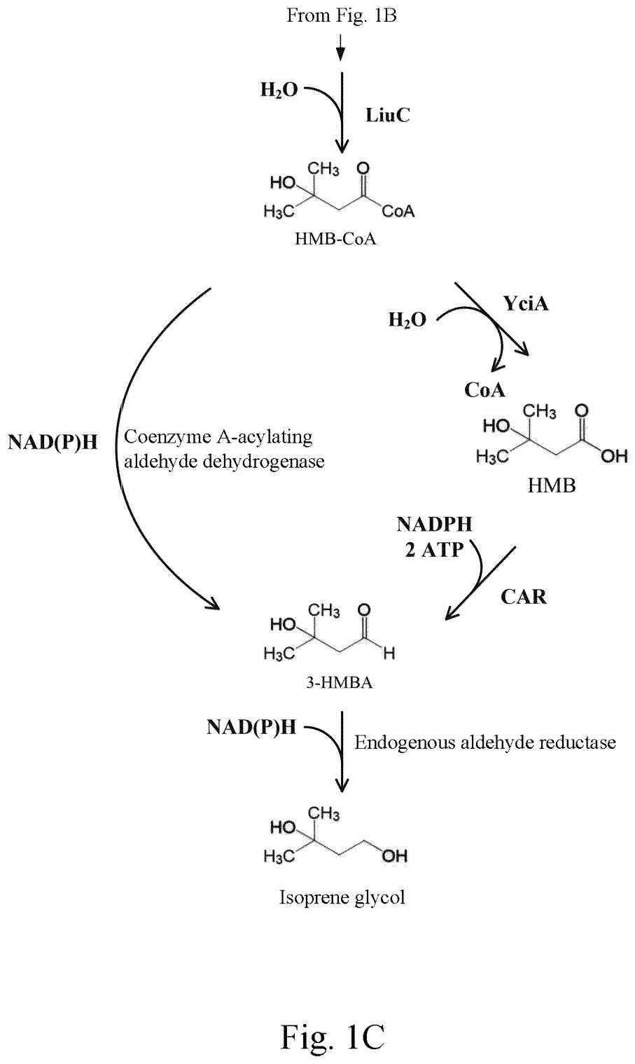

Referring to FIG. 1A, it illustrates the biosynthetic pathway of isoprene glycol in various embodiments of the present disclosure. Also referring to FIGS. 1B and 1C, which show the structural formulas of the compounds associated with the pathway in FIG. 1A. By linking the mevalonate pathway with the branched-chain fatty acid pathway, acetyl-CoA (Ac-CoA) is extended to synthesize 3-hydroxy-3-methylbutyryl-CoA (HMB-CoA). Subsequently, two pathways are designed to reduce HMB-CoA: one involves reaction with coenzyme A-acylating aldehyde dehydrogenase, and the other involves reaction with thioesterase and carboxylic acid reductase (Car).

As shown in FIG. 1A, the biosynthetic pathway of isoprene glycol begins with acetyl-CoA. Acetyl CoA is a product of glucose glycolysis.

In the first stage of the biosynthesis of isoprene glycol, HMB-CoA is enzymatically generated from acetyl-CoA. This first stage includes converting acetyl-CoA (Ac-CoA) to acetoacetyl-CoA (AcAc-CoA) using acetyl-CoA acetyltransferase (AtoB).

Next, AcAc-CoA is converted to 3-hydroxy-3-methylglutaryl-CoA (HMG-CoA) using 3-hydroxy-3-methylglutaryl-CoA synthase (MvaS).

HMG-CoA is then converted to 3-methylglutaconyl-CoA (3-MG-CoA) using HMG-CoA dehydratase/methylcrotonyl-CoA hydratase (LiuC), which has HMG-CoA dehydratase activity.

Next, 3-MG-CoA is converted to 3-methylcrotonyl-CoA (3-MC-CoA) using methylglutaconyl-CoA (MG-CoA) decarboxylase (AibA/AibB). The MG-CoA decarboxylase includes subunit A (AibA) and subunit B (AibB).

Next, 3-MC-CoA is converted to HMB-CoA using the methylcrotonyl-CoA hydratase activity of LiuC.

Then, HMB-CoA can be converted to 3-methyl-3-hydroxybutyraldehyde (3-HMBA) via two pathways: the second (A) stage or the second (B) stage.

In the second (A) stage, HMB-CoA is reduced to produce 3-HMBA via coenzyme A-acylating propionaldehyde dehydrogenase.

In the second (B) stage, the conversion occurs in two steps: first, HMB-CoA is hydrolyzed to 3-hydroxy-3-methylbutyric acid (HMB) by thioesterase (YciA, also known as acyl-CoA thioesterase); then, HMB is reduced to 3-HMBA by carboxylic acid reductase (CAR).

Next, in the third stage, 3-HMBA is converted to isoprene glycol (ISPG) by aldehyde reductase.

When the second (A) stage is used to reduce HMB-CoA to produce 3-HMBA in one step, the reaction equations for the pathway from glucose to the production of acetyl-CoA (P1) and the pathway from acetyl-CoA to the production of isoprene glycol (P2) are as follows:

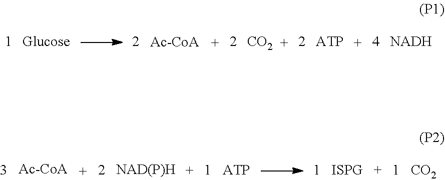

Therefore, the theoretical yield of the net reaction for pathways (P1) and (P2) is:

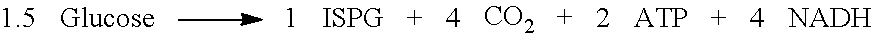

When the second (B) stage is used to convert HMB-CoA to 3-HMBA in a two-step manner, the reaction equations for the pathway from glucose to the production of acetyl-CoA (P1) and the pathway from acetyl-CoA to the production of isoprene glycol (P2) are as follows:

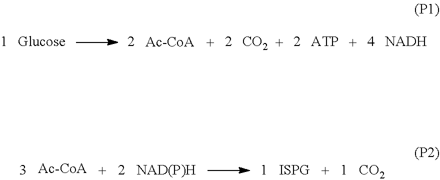

Therefore, the theoretical yield of the net reaction for pathways (P1) and (P2) is:

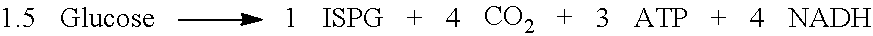

In some embodiments, the biosynthesis of isoprene glycol can be carried out through a combination of enzymes associated with each step.

In some embodiments, the biosynthesis of isoprene glycol can be carried out in a cell-free reaction system. That is, it can be conducted using a combination of purified or partially purified enzymes, or cell extracts in a reactor.

In other embodiments, the biosynthesis of isoprene glycol can be carried out through fermentation using microorganisms.

In some embodiments, the enzymes used can be naturally occurring enzymes or derived from naturally occurring enzymes, for example, through the introduction of mutations or other modifications, such as changes to or improvements in the enzyme's activity, stability, etc.

Because natural microorganisms, such as Escherichia coli (E. coli), cannot utilize carbohydrates as a carbon source to produce isoprene glycol, embodiments of the present disclosure utilizes genetic engineering techniques (such as DNA recombination technology) to transfect specific genes into the microorganisms.

In some embodiments, a combination of genetic vectors is prepared, the combination including one or more genetic vectors, such as plasmids. Each vector contains at least one sequence encoding a heterologous enzyme in the biosynthesis pathway of isoprene glycol. The combination of gene vectors contains sequences encoding multiple heterologous enzymes in the biosynthesis pathway of isoprene glycol.

In some embodiments, the enzymes associated with the metabolic pathway are transferred to microorganisms (as host cells) via gene vectors to create the transgenic microorganisms. The microorganisms may be, for example, prokaryotic bacteria (such as E. coli), cyanobacteria, filamentous fungi, or yeast.

In some embodiments, microorganisms may undergo genetic modification by introducing one or more nucleic acid molecules, which contain nucleic acid sequences which encode one or more enzymes involved in the biosynthesis pathway of isoprene glycol. In some embodiments, the introduced nucleic acid molecules may stably integrate into the genome of the microorganisms. In other embodiments, the introduced nucleic acid molecules may exist outside the genome, such as on plasmids within the microorganism.

In some embodiments, the metabolic pathways in the transgenic microorganisms include the acetyl-CoA generation pathway, the pathway generating HMB-CoA from acetyl-CoA (the first stage), the pathway reducing HMB-CoA to produce 3-HMBA (the second (A) stage) or hydrolyzing HMB-CoA to HMB followed by reducing HMB to 3-HMBA (the second (B) stage), and the pathway reducing 3-HMBA to isoprene glycol (the third stage).

In some embodiments, the transgenic microorganisms express the enzymes of the second (A) stage pathway but do not express the enzymes of the second (B) stage pathway. In other embodiments, the transgenic microorganisms express the enzymes of the second (B) stage pathway but do not express the enzymes of the second (A) stage pathway. In yet other embodiments, the transgenic microorganisms express the enzymes of both the second (A) stage pathway and the second (B) stage pathway.

In some embodiments, the genome of the microorganism has an endogenous acetyl-CoA generation pathway. In some embodiments, multiple enzymes related to the first stage pathway, and the second (A) stage pathway or the second (B) stage pathway are heterologously expressed in the host cell. In some embodiments, the genome of the microorganism has an endogenous aldehyde reductase associated with the third stage pathway. In some embodiments, the microorganism may contain heterologously expressed aldehyde reductase to facilitate the reactions of the third stage pathway.

In some embodiments, the transgenic microorganism has a first set of heterologous genes, as well as a second set of heterologous genes or a third set of heterologous genes. The first set of heterologous genes is configured to express enzymes of the first stage pathway. The second set of heterologous genes is configured to express enzymes of the second (A) stage pathway, and the third set of heterologous genes is configured to express enzymes of the second (B) stage pathway.

In some embodiments, the first set of heterologous genes and the second set of heterologous genes (or the third set of heterologous genes) may be located in a same expression plasmid in the transgenic microorganism. In other embodiments, the first set of heterologous genes and the second set of heterologous genes (or the third set of heterologous genes) may be located in two or more compatible expression plasmids.

In some embodiments, the transgenic microorganism is introduced into a bioreactor, which contains carbon source substrates and culture medium suitable for the growth of the transgenic microorganism, and is maintained at an appropriate temperature range for cultivation.

In some embodiments, since the biosynthetic isoprene glycol is synthesized via coenzyme A, vitamin B5 (pantothenic acid) may be added to the culture medium during the cultivation of the transgenic microorganism to activate pathways related to coenzyme A production. In some embodiments, the transgenic microorganism may also include heterologously expressed pantothenate kinase, such as CoaA, and vitamin B5 may be added to the culture medium to supplement coenzyme A, thereby increasing the intracellular concentration of coenzyme A.

In some embodiments, the transgenic microorganism may also include heterologously expressed enzymes that activate carboxylic acid reductase, such as 4′-phosphopantetheinyl transferase, e.g., sfp or entD.

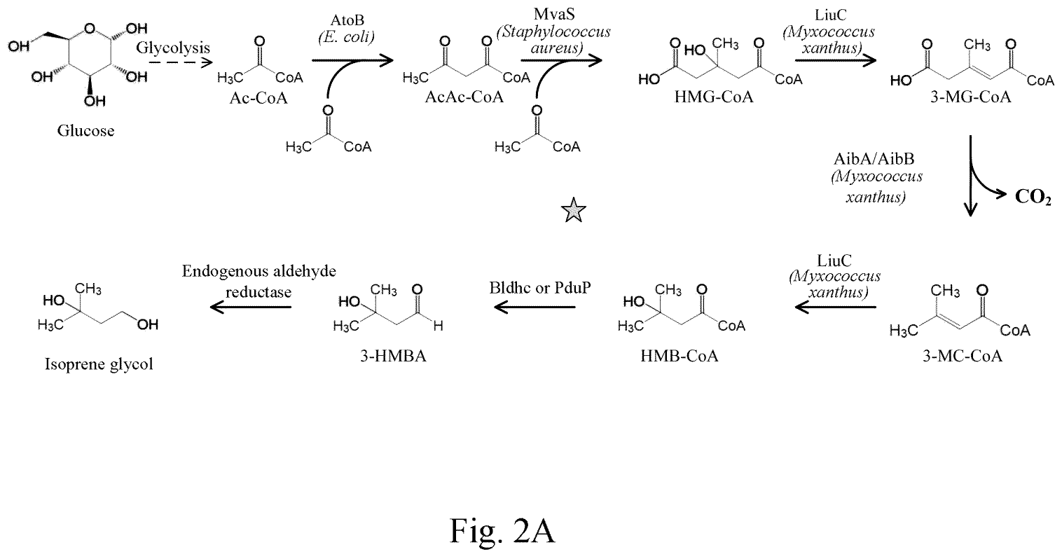

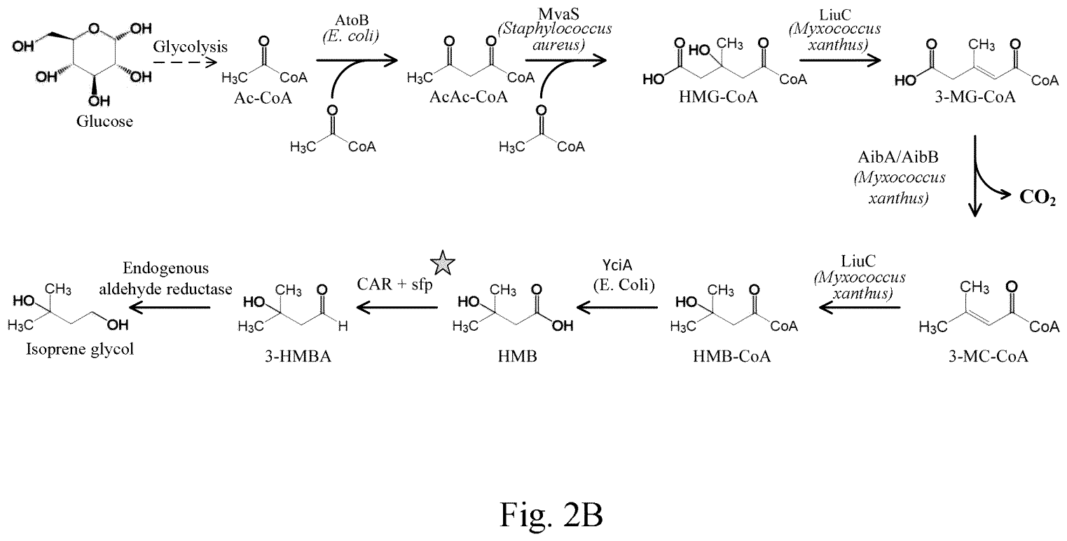

Referring to FIG. 2A, which shows the metabolic pathway in the transgenic microorganism according to some embodiments and the sources of the heterologously expressed enzymes. In FIG. 2A, HMB-CoA is converted to 3-HMBA via the second (A) stage pathway.

In FIG. 2A, the transgenic microorganism contains endogenous enzymes associated with glycolysis. Acetyl-CoA is converted to AcAc-CoA via AtoB from E. coli. AcAc-CoA is converted to HMG-CoA via MvaS from Staphylococcus aureus. HMG-CoA is converted to 3-MG-CoA via LiuC from Myxococcus xanthus. 3-MG-CoA is converted to 3-MC-CoA via AibA and AibB from Myxococcus xanthus. 3-MC-CoA is converted to HMB-CoA via LiuC from Myxococcus xanthus. HMB-CoA is converted to 3-HMBA via Bldhc or PduP from different bacteria species. 3-HMBA is converted to isoprene glycol via endogenous aldehyde reductase.

Referring to FIG. 2B, which shows the metabolic pathway in the transgenic microorganism according to some embodiments and the sources of the heterologously expressed enzymes. In FIG. 2B, HMB-CoA is converted to 3-HMBA via the second (B) stage pathway.

In FIG. 2B, the transgenic microorganism contains endogenous enzymes associated with glycolysis. Acetyl-CoA is converted to AcAc-CoA via AtoB from E. coli. AcAc-CoA is converted to HMG-CoA via MvaS from Staphylococcus aureus. HMG-CoA is converted to 3-MG-CoA via LiuC from Myxococcus xanthus. 3-MG-CoA is converted to 3-MC-CoA via AibA and AibB from Myxococcus xanthus. 3-MC-CoA is converted to HMB-CoA via LiuC from Myxococcus xanthus. HMB-CoA is converted to HMB via YciA from E. coli. The conversion of HMB to 3-HMBA may be achieved through different carboxylic acid reductases (CAR) and enzymes that activate carboxylic acid reductases (e.g., sfp). 3-HMBA is converted to isoprene glycol via endogenous aldehyde reductase.

In some embodiments, the acetyl-CoA acetyltransferase (AtoB) expressed in the transgenic microorganism belong to the EC: 2.3.1.9 family of enzymes. In some embodiments, the polypeptide of the acetyl-CoA acetyltransferase (AtoB) heterologously expressed in the transgenic microorganism has at least 80% identity to SEQ ID NO: 1 and exhibits the activity of acetyl-CoA acetyltransferase (EC: 2.3.1.9). The sequence of the AtoB may have 80%, 85%, 90%, 95%, or 100% identity to SEQ ID NO: 1.

In some embodiments, the MvaS expressed in the transgenic microorganism belongs to the EC: 2.3.3.10 family of enzymes. In some embodiments, the polypeptide of the MvaS heterologously expressed in the transgenic microorganism has at least 70% identity to SEQ ID NO: 2 and exhibits the activity of HMG-CoA synthase (EC: 2.3.3.10). The sequence of the MvaS may have 70%, 75%, 80%, 85%, 90%, 95%, or 100% identity to SEQ ID NO: 2.

In some embodiments, the LiuC expressed in the transgenic microorganism belongs to the EC: 4.2.1.18 family of enzymes. In some embodiments, the polypeptide of the LiuC heterologously expressed in the transgenic microorganism has at least 70% identity to SEQ ID NO: 3 and exhibits the activity of Mc-CoA hydratase (EC 4.2.1.18). The sequence of the LiuC may have 70%, 75%, 80%, 85%, 90%, 95%, or 100% identity to SEQ ID NO: 3.

In some embodiments, the heterologously expressed methylglutaconyl-CoA (MG-CoA) decarboxylase in the transgenic microorganism includes MG-CoA decarboxylase subunit A (AibA) and MG-CoA decarboxylase subunit B (AibB). The polypeptide of the AibA has at least 70% identity to SEQ ID NO: 4 and exhibits the activity of MG-CoA decarboxylase. The sequence of the AibA may be 70%, 75%, 80%, 85%, 90%, 95%, or 100% identity to SEQ ID NO: 4. Additionally, the polypeptide of the AibB has at least 70% identity to SEQ ID NO: 5 and exhibits the activity of MG-CoA decarboxylase. The sequence of the AibB may have 70%, 75%, 80%, 85%, 90%, 95%, or 100% identity to SEQ ID NO: 5.

In some embodiments, the pantothenate kinase expressed in the transgenic microorganism belongs to the EC: 2.7.1.33 family of enzymes. In some embodiments, the polypeptide of the pantothenate kinase heterologously expressed in the transgenic microorganism has at least 60% identity to SEQ ID NO: 10 and exhibits the activity of pantothenate kinase (EC 2.7.1.33). The sequence of the pantothenate kinase may have 60%, 65%, 70%, 75%, 80%, 85%, 90%, 95%, or 100% identity to SEQ ID NO: 10.

In some embodiments, the coenzyme A-acylating aldehyde dehydrogenase expressed in the transgenic microorganism belongs to the EC: 1.2.1.87 family of enzymes. In some embodiments, the polypeptide of the coenzyme A-acyl aldehyde dehydrogenase that is heterologously expressed in the transgenic microorganism has at least 60% identity to SEQ ID NO: 7, SEQ ID NO: 12, or SEQ ID NO: 13 and exhibits the activity of coenzyme A-acyl aldehyde dehydrogenase (EC: 1.2.1.87). The sequence of the coenzyme A-acyl aldehyde dehydrogenase may have 60%, 65%, 70%, 75%, 80%, 85%, 90%, 95%, or 100% identity to SEQ ID NO: 7, SEQ ID NO: 12, or SEQ ID NO: 13.

In some embodiments, the thioesterase expressed in the transgenic microorganism belongs to the EC: 3.1.2.-family of enzymes. In some embodiments, the heterologously expressed polypeptide of the thioesterase (YciA) in the transgenic microorganism has at least 60% identity to SEQ ID NO: 6 and possesses the activity of thioesterase (EC: 3.1.2.-). The sequence of the thioesterase may have 60%, 65%, 70%, 75%, 80%, 85%, 90%, 95%, or 100% identity to SEQ ID NO: 6.

In some embodiments, the carboxylic acid reductase (CAR) expressed in the transgenic microorganism belongs to the EC: 1.2.1.30 family of enzymes. In some embodiments, the heterologously expressed polypeptide of the CAR in the transgenic microorganism has at least 60% identity to SEQ ID NO: 8, SEQ ID NO: 14, or SEQ ID NO: 15 and has the activity of carboxylic acid reductase (EC: 1.2.1.30). The sequence of the CAR may have 60%, 65%, 70%, 75%, 80%, 85%, 90%, 95%, or 100% identity to SEQ ID NO: 8, SEQ ID NO: 14, or SEQ ID NO: 15.

In some embodiments, the 4′-phosphopantetheinyl transferase expressed in the transgenic microorganism belongs to the EC: 2.7.8.7 family of enzymes. In some embodiments, the heterologously expressed polypeptide of the 4′-phosphopantetheinyl transferase has at least 60% identity to SEQ ID NO: 9 and has the activity of 4′-phosphopantetheinyl transferase (EC: 2.7.8.7). The sequence of the 4′-phosphopantetheinyl transferase may have 60%, 65%, 70%, 75%, 80%, 85%, 90%, 95%, or 100% identity to SEQ ID NO: 9.

In some embodiments, the aldehyde reductase expressed in the transgenic microorganism belongs to the EC: 1.1.1.2 family of enzymes. In some embodiments, the heterologously expressed aldehyde reductase in the transgenic microorganism has at least 60% identity to SEQ ID NO: 11 and has the activity of aldehyde reductase (EC: 1.1.1.2). The sequence of the heterologous aldehyde reductase may have 60%, 65%, 70%, 75%, 80%, 85%, 90%, 95%, or 100% identity to SEQ ID NO: 11.

The sequences in the sequence listings of the present disclosure are reference amino acid sequences. In some embodiments, the sequence of each enzyme may include amino acids other than those essential for function (e.g., catalytic activity of the protein), or subunit folding or structure may be missing or replaced by insertions. Alternatively, essential amino acids may be conservatively substituted to achieve an effect in which the enzymatic activity of the reference sequence or derived molecules is retained.

In various embodiments of the present disclosure, the strains of the transgenic microorganisms and the materials and reagents used in the methods are commercially available.

The following experiment examples provide various transgenic microorganisms and related experimental test results.

### Experiment Example 1

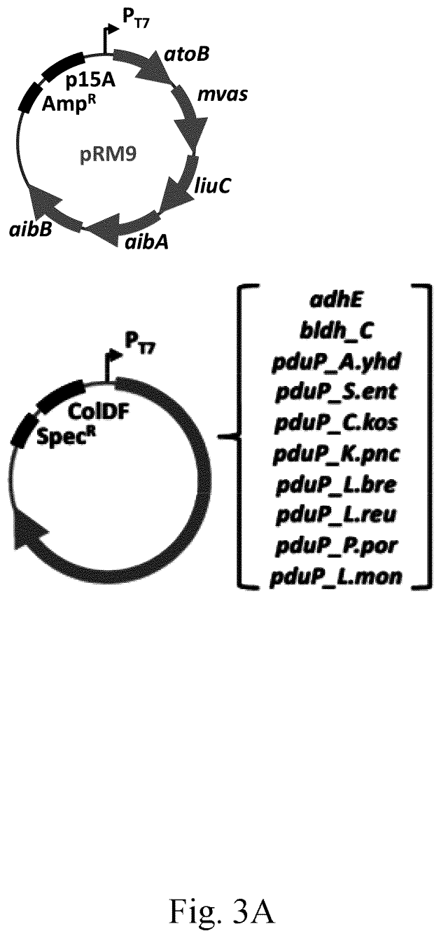

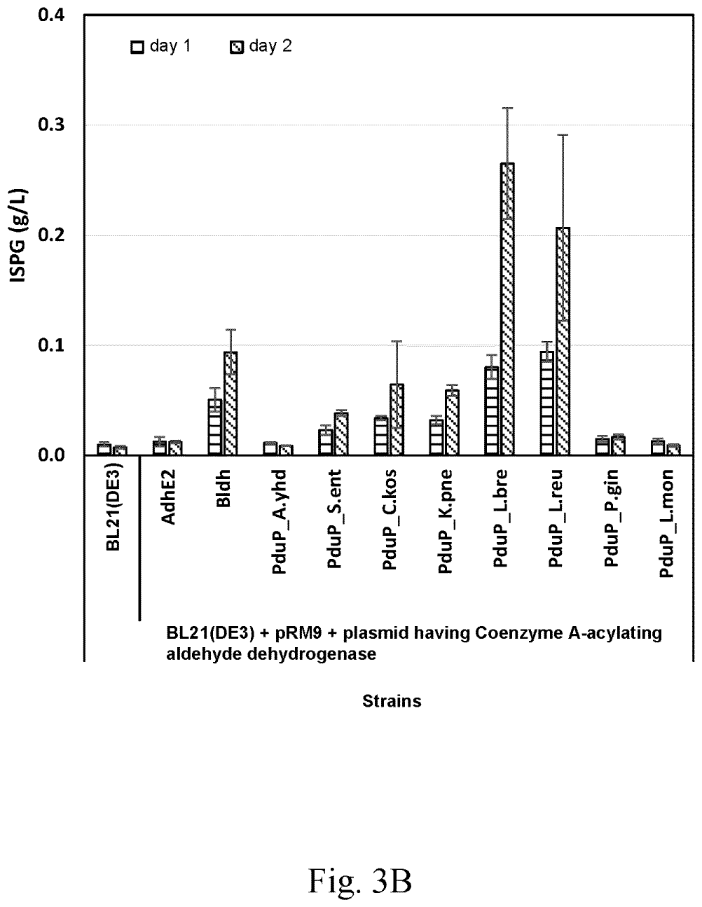

Referring to FIG. 3A, a dual plasmid system was designed, which includes the first plasmid (pRM9 plasmid) and the second plasmid. The first plasmid expresses the pathway from acetyl-CoA to the production of HMB-CoA (i.e., the first stage shown in FIG. 1A), which includes atoB, mvaS, liuC, aibA, and aibB. The second plasmid expresses CoA-aldehyde dehydrogenase, and the enzyme encoded in this region of the plasmid are indicated in parentheses, meaning that in this experimental example, each of the strains of the transgenic microorganisms tested contains a respective second plasmid. Each different second plasmid encodes a different dehydrogenase. In Experiment Example 1, 10 different dehydrogenases were tested, including: aldehyde-alcohol dehydrogenase (adhE), CoA-acylating butyraldehyde dehydrogenase (bldh), and CoA-acylating propionaldehyde dehydrogenase (PduP) from 8 different bacteria species.

The AdhE originates from Clostridium acetobutylicum, and the Bldh originates from Clostridium beijerinckii. PduP respectively originates from Aeromonas hydrophila (A. hyd), Salmonella enterica (S. ent), Citrobacter koseri (C. kos), Klebsiella pneumoniae (K. pne), Lactobacillus brevis (L. bre), Lactobacillus reuteri (L. reu), Porphyromonas gingivalis (P. gin), and Listeria monocytogenes (L. mon).

FIG. 3B shows the yields of isoprene glycol after culturing the strains having the dual plasmid system depicted in FIG. 3A. Expression of Bldh and PduP from S. ent, C. kos, K. pne, L. bre, and L. reu successfully converted glucose to isoprene glycol within a day, with yields ranging from approximately 0.05 to 0.25 g/L. Among these strains, the PduP from Lactobacillus genus produced a relatively higher amount of isoprene glycol.

### Experiment Example 2

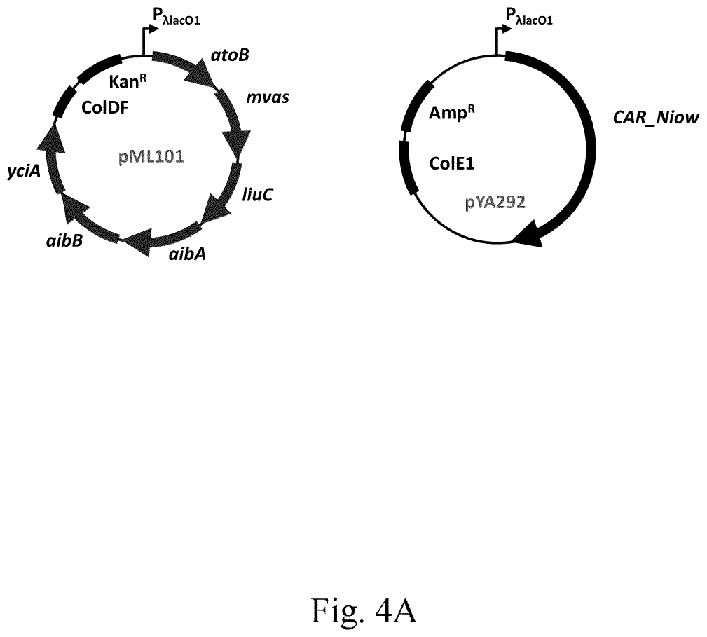

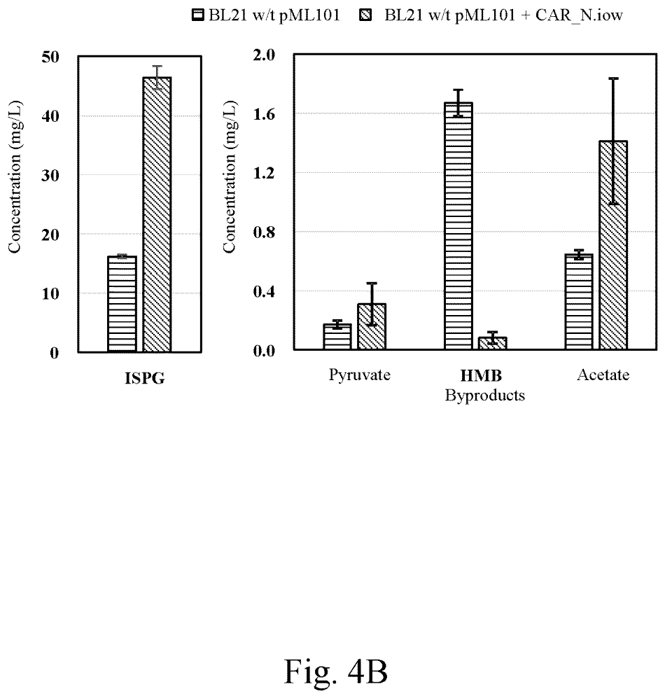

Referring to FIG. 4A, strains producing HMB were used to produce HMB, and the difference in isoprene glycol yield was tested with and without expressing carboxylic acid reductase (CAR) in the strains. The HMB-producing strains were BL21(DE3) transformed with the pML101 plasmid, expressing AtoB and YciA from E. coli, MvaS from Staphylococcus aureus, and LiuC and AibA/AibB from Myxococcus xanthus, hereafter referred to as the “base strain.” Furthermore, a second plasmid was used to express the ATP/NADPH-dependent carboxylic acid reductase gene from Nocardia iowensis (i.e., CAR_Niow).

FIG. 4B shows the concentrations of isoprene glycol (ISPG) and by-products (pyruvate, HMB, and acetate) in the culture medium. The production of isoprene glycol was measured in the HMB-producing strains with and without the expression of carboxylic acid reductase (CAR) over a 24-hour period of cultivation. It was observed that the HMB-producing strain expressing CAR produced approximately 45 mg/L of isoprene glycol, three times the yield of the base HMB-producing strain. Correspondingly, the HMB precursor was reduced in the CAR-expressing HMB-producing strain, indicating that HMB was converted by CAR into HMB-aldehyde.

### Experiment Example 3

In this example, a bio-prospecting experiment was conducted using a single plasmid system: CARs from various microbial species were expressed, and 40 mM of HMB was added to the medium. Differences in isoprene glycol production by E. coli strain BL21 (DE3) served as a basis for analyzing the intracellular CAR activity.

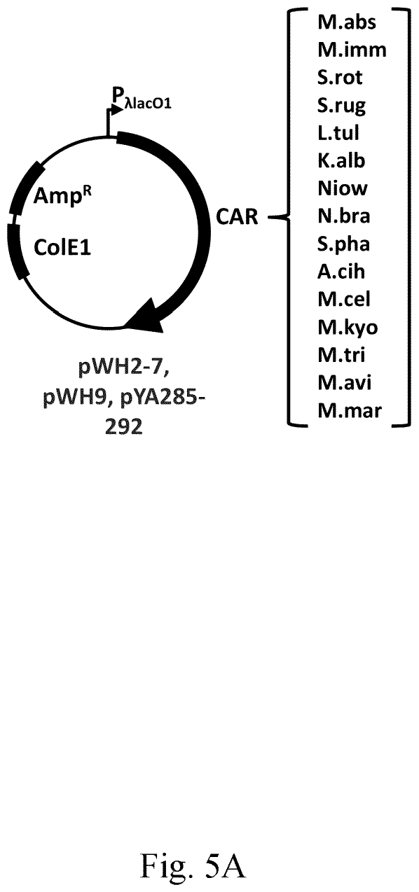

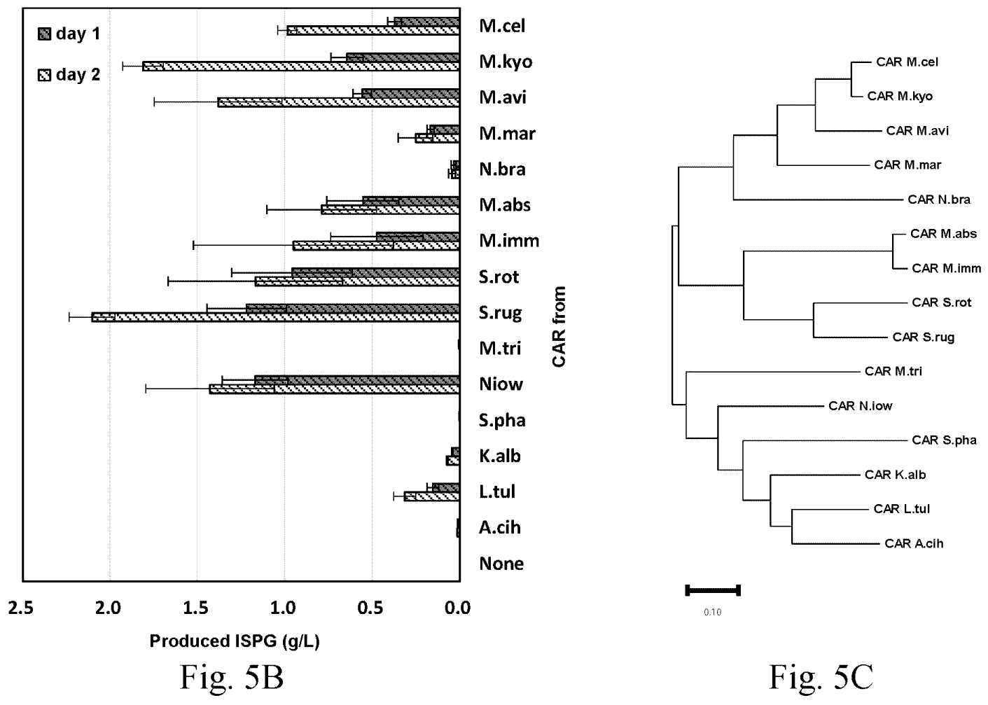

FIG. 5A illustrates the plasmid design, and each of the plasmids expresses CAR from a respective bacterial species. The 15 tested bacteria specieds included Mycobacterium abscessus (M. abs), Mycobacterium immunogenum (M. imm), Segniliparus rotundus (S. rot), Segniliparus rugosus (S. rug), Longimycelium tulufanense (L. tul), Kutzneria albida (K. alb), Nocardia brasiliensis (N. bra), Saccharopolyspora phatthalungensis (S. pha), Amycolatopsis cihanbeyliensis (A. cih), Mycobacterium celatum (M. cel), Mycobacterium kyorinense (M. kyo), Mycobacterium triplex (M. tri), Mycobacterium avium (M. avi), Mycobacterium marinum (M. mar), and Nocardia iowensis (N. iow).

As shown in FIG. 5B, in this test, 9 CAR isoenzymes from these bacteria species were found to have HMB reduction activity, specifically from bacteria species M. cel, M. kyo, M. avi, M. mar, M. abs, M. imm, S. rot, S. rug, and N. iow. Compared to strains without CAR expression, strains overexpressing CAR from S. rug, M. kyo, and M. avi produced 1.5 to 2 g/L of isoprene glycol. Using the CAR from Nocardia iow (N. iow) as a baseline (which is also used in patent CN111349644A), the CAR from S. rug (CAR_S.rug) and the CAR from M. kyo (CAR_M.kyo) exhibited higher HMB conversion efficiencies. Therefore, the embodiments of the present disclosure have developed CAR enzymes that are more suitable for producing isoprene glycol.

Table 1 below shows the sequences of 14 other carboxylic acid reductases compared to the CAR_S.rug, which has the best conversion efficiency with isoprene glycol. The identities and positives of the amino acid sequences were calculated using the NCBI Blast website (https://blast.ncbi.nlm.nih.gov/Blast.cgi NCBI Blast).

TABLE 1
Sequence Identities and Positives Using
CAR_S.rug as the Reference Sequence

| Reference CAR | Compared CAR | | |
|---|---|---|---|
| from | from | Identities | Positives |
| CAR_S.rug | CAR_M.abs | 61% | 75% |
| | CAR_M.imm | 61% | 75% |
| | CAR_S.rot | 73% | 85% |
| | CAR_L.tul | 59% | 74% |
| | CAR_K.alb | 59% | 73% |
| | CAR_N.iow | 59% | 73% |
| | CAR_N.bra | 55% | 69% |
| | CAR_S.pha | 55% | 71% |
| | CAR_A.cih | 58% | 72% |
| | CAR_M.cel | 59% | 73% |
| | CAR_M.kyo | 59% | 73% |
| | CAR_M.tri | 56% | 72% |
| | CAR_M.avi | 57% | 72% |
| | CAR_M.mar | 57% | 71% |

FIG. 5C shows a phylogenetic tree of the sequence alignment for the tested CARs. In FIG. 5C, the scale bar of “0.1” represents a basic unit of branch length distance on the phylogenetic tree, indicating the degree of divergence between adjacent branches. The phylogenetic tree in FIG. 5C was constructed using the Maximum Likelihood method, and it reflects the topological analysis of the similarity and dissimilarity of the amino acid sequences of all CARs, which may be associated with the varying expression levels of CARs within E. coli. However, CAR activities are not only related to the expression levels but also to the identity of the active site sequences of CARs. This suggests that although the overall sequence identity may not differ significantly, there is still a possibility of varying levels of activity among different CARS.

By comparing FIG. 5B and FIG. 5C, it can be observed that the 15 CARS clustered based on the phylogenetic tree can be divided into two clusters that can convert HMB to ISPG: one cluster includes CAR_M.cel, CAR_M.kyo, and CAR_M.avi; the other cluster includes CAR_M.abs, CAR_M.imm, CAR_S.rug, and CAR_S.rot.

### Experiment Example 4

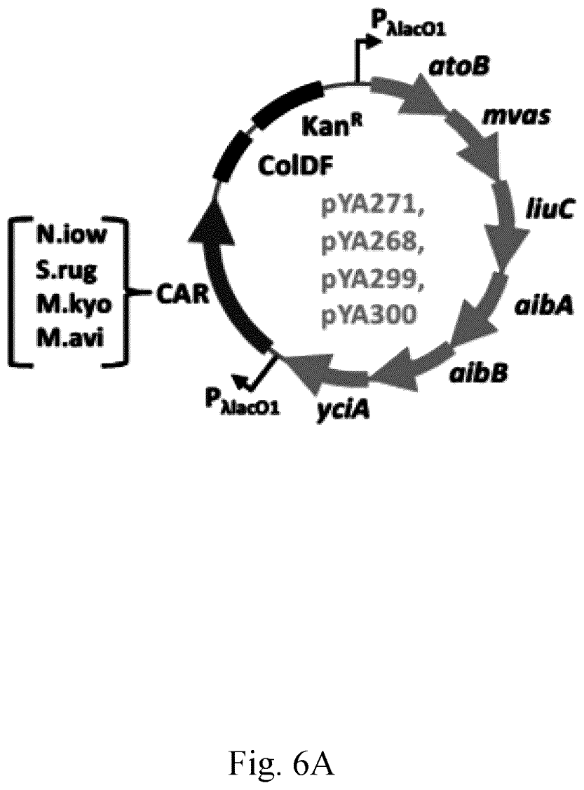

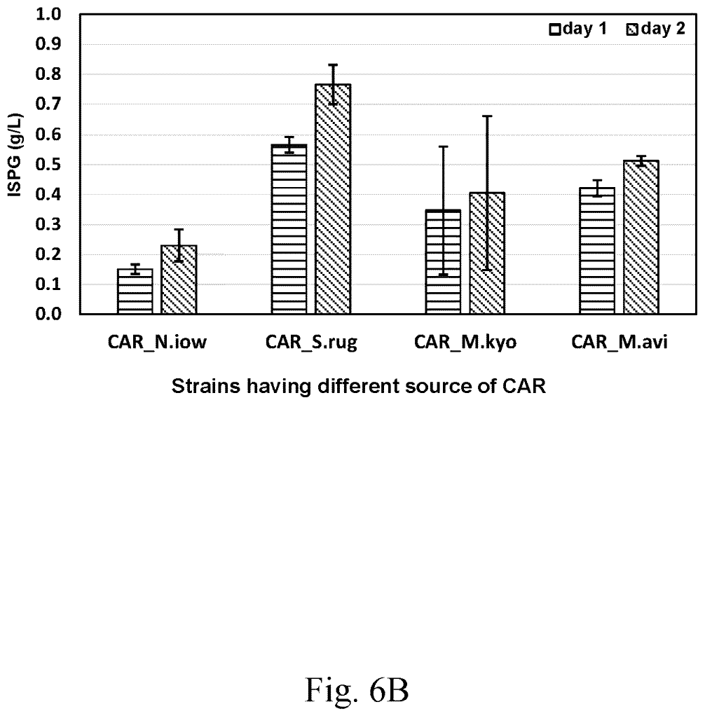

In this experiment, three high-efficiency CAR candidate enzymes obtained from the bio-prospecting were integrated with the HMB production module into a single plasmid expression system. As shown in FIG. 6A, the plasmid design is presented, with different CARs in the plasmids respectively originating from N. iow (CAR_N.iow), S. rug (CAR_S.rug), M. kyo (CAR_M.kyo), and M. avi (CAR_M.avi).

A two-day production fermentation test was then conducted and compared with the CAR from Nocardia iowensis (N. iow) initially used in the present disclosure. The results are shown in FIG. 6B. It can be seen that in the single plasmid system, the production of isoprene glycol using CAR_N.iow increased approximately threefold (from 45 mg/L to 150 mg/L). All three high-efficiency CAR candidate enzymes enabled the strain to produce more isoprene glycol, with CAR_S.rug (plasmid pYA268) achieving the highest production efficiency, yielding 560 to 770 mg/L of isoprene glycol. This result surpasses the highest yield of 300 mg/L reported in the previous literature and patents (Reference: Liu, Y., W. Wang, and A. P. Zeng (2022). “Biosynthesizing structurally diverse diols via a general route combining oxidative and reductive formations of OH-groups.” Nat Commun 13 (1): 1595.). Therefore, the embodiments of the present disclosure demonstrate significant improvements in isoprene glycol production via biosynthesis.

### Experiment Example 5

In this experiment example, a second plasmid was constructed to enhance the efficiency of HMB reduction to isoprene glycol. On one hand, an aldehyde reductase, YqhD (from E. coli), was co-expressed to improve the catalytic efficiency of converting HMB-aldehyde to isoprene glycol. On the other hand, an activating enzyme of CAR_S.rug was expressed to activate CAR_S.rug. The activating enzyme is configured for consuming coenzyme A (CoA). The tested activating enzymes were 4′-phosphopantetheinyl transferase Sfp from Bacillus subtilis and 4′-phosphopantetheinyl transferase EntD from the same species as CAR_S.rug.

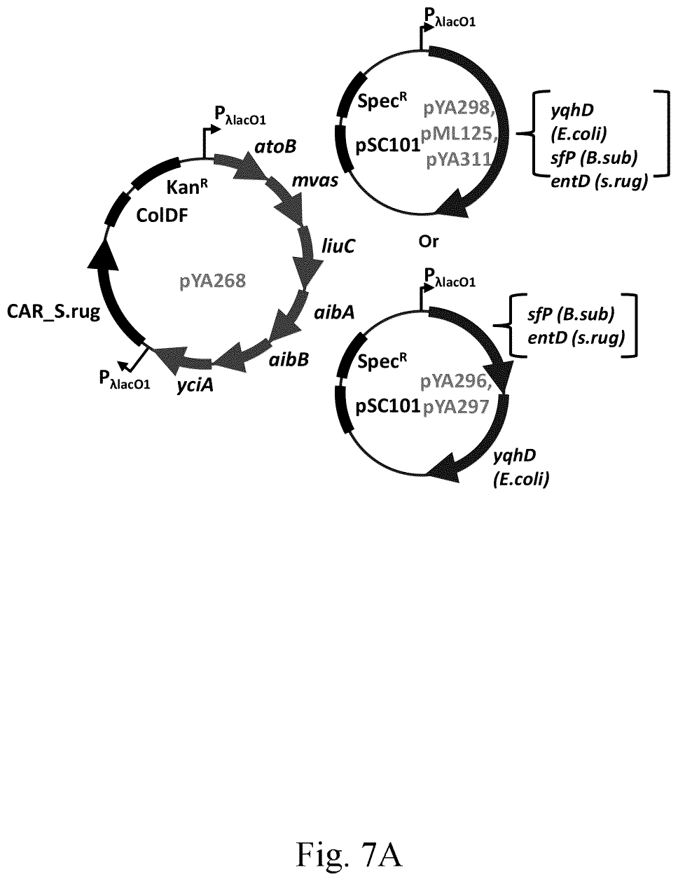

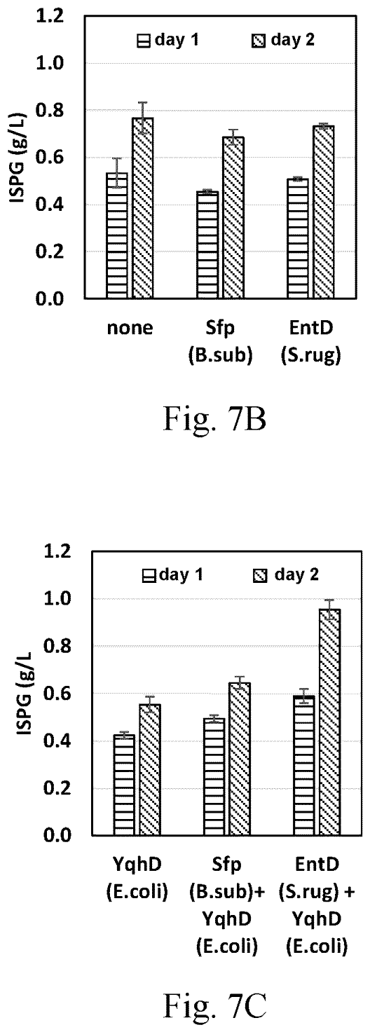

As shown in FIG. 7A, based on the three enzymes to be expressed, five plasmids were constructed: pYA298 (yqhD), pML125 (sfp), pYA311 (entD), pYA296 (sfp+yqhD), and pYA297 (entD+yqhD), which were co-expressed in the isoprene glycol production strain (which has the pYA268 plasmid).

Referring to FIGS. 7B and 7C, which show the isoprene glycol production of the dual plasmid system constructed in FIG. 7A over a period of 2 days. As shown in FIG. 7B, expressing Sfp individually and EntD individually did not effectively increase the isoprene glycol yield. However, as shown in FIG. 7C, co-expression of EntD and YqhD raised the isoprene glycol yield to 950 mg/L.

### Experiment Example 6

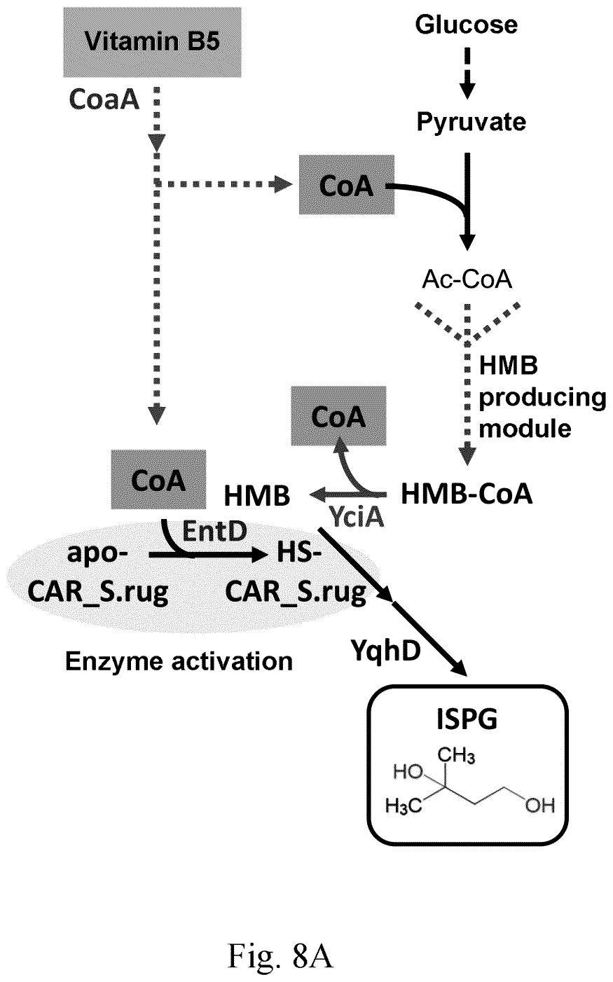

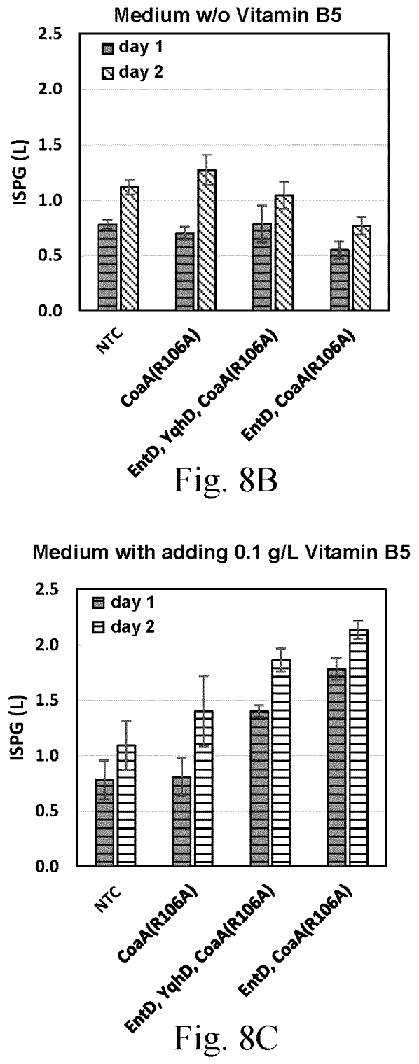

FIG. 8A shows a schematic of the metabolic pathway from glucose to isoprene glycol and the reactions involving coenzyme A (CoA) in this pathway. As shown in FIG. 8A, adding vitamin B5 (pantothenic acid) can promote CoA production in this pathway.

In this experiment example, the strategy to enhance intracellular coenzyme A carbon flux involved expressing pantothenate kinase (CoaA) in a dual plasmid system to confer resistance to CoA feedback inhibition, with vitamin B5 (pantothenic acid) added to the culture medium.

Referring to FIGS. 8B and 8C, which show the isoprene glycol production of different strains having a dual plasmid system after 2 days of cultivation. It can be seen that only the strains expressing CoaA and supplemented with vitamin B5 can enhance isoprene glycol production, with increases ranging from 20% to 90%, and the highest production can reach up to 2.1 g/L.

### Experiment Example 7

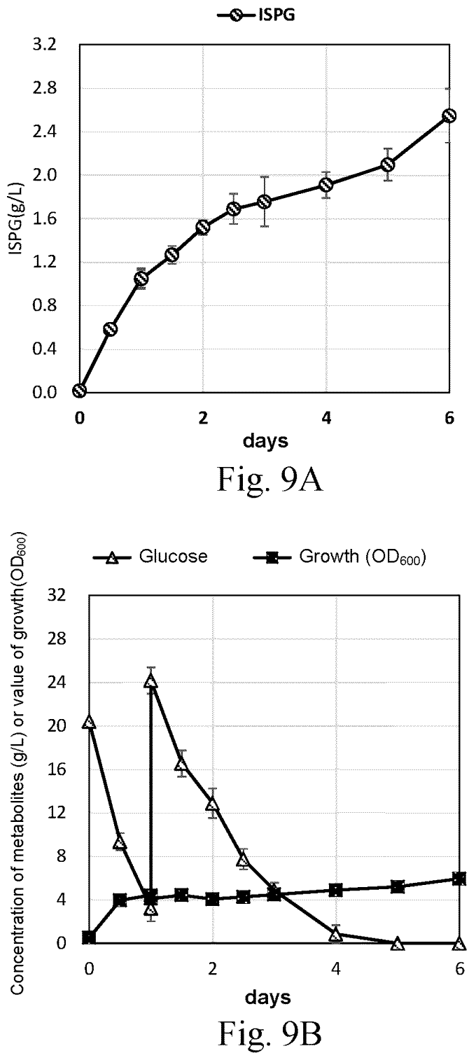

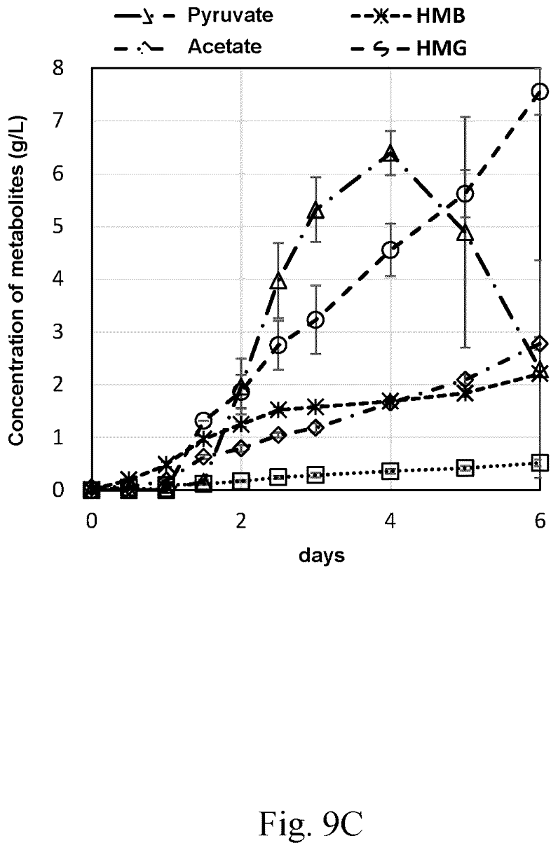

In this experiment example, the tested strain was E. coli expressing AtoB, Mvas, LiuC, AibA/AibB, YciA, CAR_S.rug, and co-expressing EntD and CoaA. The strain was cultivated in a flask for an extended period (up to 6 days). Glucose was added on the first day, vitamin B5 was added every two days, and the pH of the culture medium was adjusted to 7 at each sampling point. The results are shown in FIGS. 9A to 9C.

FIG. 9A shows the yield of isoprene glycol, FIG. 9B shows the glucose concentration and the growth of the strain (measured via OD600 absorbance), and FIG. 9C shows the yield of various metabolites. It can be observed that 2.5 g/L of isoprene glycol can be produced over 6 days of cultivation. This result demonstrates high potential for application to develop fermentation processes.

The biosynthetic pathway of isoprene glycol provided in the embodiments of the present disclosure allows for the synthesis of isoprene glycol from renewable resources, e.g., glucose. Additionally, in the embodiments, the transgenic microorganisms can use glucose as a carbon source to produce isoprene glycol through fermentation.

In some embodiments, isoprene glycol yield can be increased by bio-prospecting for related enzymes (e.g., CAR proteins) from different microorganisms, eliminating competing pathways, co-expressing enzymes to activate relevant enzymes (such as CAR-activating enzymes), or introducing strategies to enhance CoA supply. In some embodiments, with the combination of these above strategies, engineered microorganisms (e.g., E. coli) can produce 2.1 g/L of isoprene glycol within 2 days, far exceeding the 300 mg/L yield of previous technologies using a amino acid related pathway.

Although the present disclosure has been described above with embodiments and examples, it is not intended to limit the scope of the present disclosure. Any person skilled in the art may make various modifications and adjustments without departing from the spirit and scope of the present disclosure. Therefore, the protection scope of the present disclosure shall be defined by the claims appended hereafter.

What is claimed is:

1. A method for producing isoprene glycol, comprising:
enzymatically generating 3-hydroxy-3-methylbutyryl-CoA (HMB-CoA) from acetyl-CoA (Ac-CoA);
enzymatically generating 3-methyl-3-hydroxybutyrylaldehyde (3-HMBA) from the HMB-CoA; and
enzymatically converting the 3-HMBA to isoprene glycol (ISPG);
wherein the enzymatically generating 3-HMBA from the HMB-CoA comprises:
enzymatically reducing the HMB-CoA to the 3-HMBA, or enzymatically hydrolyzing the HMB-CoA to 3-hydroxy-3-methylbutyric acid (HMB) and then enzymatically reducing the HMB to the 3-HMBA.

2. The method for producing isoprene glycol of claim 1, wherein the enzymatically generating HMB-CoA from Ac-CoA comprises:
enzymatically converting the Ac-CoA to acetoacetyl-CoA (AcAc-CoA);
enzymatically converting the AcAc-CoA to 3-hydroxy-3-methylglutaryl coenzyme A (HMG-CoA);
enzymatically converting the HMG-CoA to 3-methylglutaconyl-CoA (3-MG-CoA);
enzymatically converting the 3-MG-CoA to 3-methyl crotonyl-CoA (3-MC-CoA); and
enzymatically converting the 3-MC-CoA to the HMB-CoA.

3. An enzyme composition for producing isoprene glycol, comprising:
a first group comprising: acetyl-CoA acetyltransferase (AtoB), 3-hydroxy-3-methylglutaryl coenzyme A (HMG-CoA) synthase (MvaS), HMG-CoA dehydratase/methylcrotonyl-CoA (MC-CoA) hydratase (LiuC), and methylglutaconyl-CoA (MG-CoA) decarboxylase (AibA/AibB); and
a second group or a third group, wherein the second group comprises: coenzyme A-acylating aldehyde dehydrogenase, and the third group comprises thioesterase (YciA) and carboxylic acid reductase (CAR).

4. A nucleic acid composition for producing isoprene glycol, comprising:
at least one gene vector, configured to express the enzyme composition for producing isoprene glycol of claim 3.

5. A transgenic microorganism for producing isoprene glycol, comprising:
an acetyl-CoA generation pathway, producing acetyl-CoA from a carbon source;
a first stage pathway, generating 3-hydroxy-3-methylbutyryl-CoA (HMB-CoA) from the acetyl-CoA;
a second (A) stage pathway or a second (B) stage pathway, wherein the second (A) stage pathway reduces the HMB-CoA to produce 3-methyl-3-hydroxybutyrylaldehyde (3-HMBA), and the second (B) stage pathway hydrolyzes the HMB-CoA to 3-hydroxy-3-methylbutyric acid (HMB) and then reduces the HMB to 3-HMBA; and
a third stage pathway, reducing the 3-HMBA to isoprene glycol (ISPG).

6. The transgenic microorganism for producing isoprene glycol of claim 5, comprising:
a host cell, wherein a genome of the host cell encodes genes for the acetyl-CoA generation pathway;
a first set of heterologous genes, located in the host cell, configured to express enzymes of the first stage pathway; and
a second set of heterologous genes or a third set of heterologous genes, located in the host cell, wherein the second set of heterologous genes is configured to express at least one enzyme of the second (A) stage pathway, and the third set of heterologous genes is configured to express at least one enzyme of the second (B) stage pathway.

7. The transgenic microorganism for producing isoprene glycol of claim 6, wherein the enzymes of the first set of heterologous genes comprise: acetyl-CoA acetyltransferase (AtoB), 3-hydroxy-3-methylglutaryl coenzyme A (HMG-CoA) synthase (MvaS), HMG-CoA dehydratase/methylcrotonyl-CoA (MC-CoA) hydratase (LiuC), and methylglutaconyl-CoA (MG-CoA) decarboxylase (AibA/AibB).

8. The transgenic microorganism for producing isoprene glycol of claim 7, wherein:
the AtoB has at least 80% identity to SEQ ID NO: 1;
the MvaS has at least 70% identity to SEQ ID NO: 2;
the LiuC has at least 70% identity to SEQ ID NO: 3; and
the methylglutaconyl-CoA (MG-CoA) decarboxylase includes a MG-CoA decarboxylase subunit A (AibA) and a MG-CoA decarboxylase subunit B (AibB), wherein the AibA has at least 70% identity to SEQ ID NO: 4, and the AibB has at least 70% identity to SEQ ID NO: 5.

9. The transgenic microorganism for producing isoprene glycol of claim 8, wherein the first set of heterologous genes further encodes pantothenate kinase configured to supplement coenzyme A (CoA).

10. The transgenic microorganism for producing isoprene glycol of claim 9, wherein the pantothenate kinase has at least 60% identity to SEQ ID NO: 10.

11. The transgenic microorganism for producing isoprene glycol of claim 6, wherein the second set of heterologous genes comprises: coenzyme A-acylating aldehyde dehydrogenase.

12. The transgenic microorganism for producing isoprene glycol of claim 11,
wherein the coenzyme A-acylating aldehyde dehydrogenase has at least 60% identity to SEQ ID NO: 7, SEQ ID NO: 12, or SEQ ID NO: 13.

13. The transgenic microorganism for producing isoprene glycol of claim 6,
wherein the third set of heterologous genes comprises thioesterase (YciA) and carboxylic acid reductase (CAR).

14. The transgenic microorganism for producing isoprene glycol of claim 13, wherein:
the YciA has at least 60% identity to SEQ ID NO: 6; and
the CAR has at least 60% identity to SEQ ID NO: 8, SEQ ID NO: 14, or SEQ ID NO: 15.

15. The transgenic microorganism for producing isoprene glycol of claim 6, further comprising:
a heterologous gene, encoding a heterologous aldehyde reductase to facilitate the third stage pathway.

16. The transgenic microorganism for producing isoprene glycol of claim 15,
wherein the heterologous aldehyde reductase has at least 60% identity to SEQ ID NO: 11.

17. The transgenic microorganism for producing isoprene glycol of claim 6, wherein the first set of heterologous genes and the second set of heterologous genes or the third set of heterologous genes are located on a same plasmid, two plasmids, or more than two plasmids.

18. A method for producing isoprene glycol, comprising:
providing the transgenic microorganism for producing isoprene glycol of claim 5; and
cultivating the transgenic microorganism for producing isoprene glycol in a culture medium, wherein a carbon source in a culture medium comprises glucose.

19. The method for producing isoprene glycol of claim 18, further comprising:
adding vitamin B5 into the culture medium.


SEQUENCE LISTING (15 sequences)

SEQ ID NO: 1 - protein, 394 aa, Escherichia coli
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLN

SEQ ID NO: 2 - protein, 388 aa, Staphylococcus aureus
MTIGIDKINFYVPKYYVDMAKLAEARQVDPNKFLIGIGQTEMAVSPVNQDIVSMGANAAKDIITDEDKKKIGMVIVATESAVDAAKAAAVQIHNLLGIQPFARCFEMKEACYAATPAIQLAKDYLATRPNEKVLVIATDTARYGLNSGGEPTQGAGAVAMVIAHNPSILALNEDAVAYTEDVYDFWRPTGHKYPLVDGALSKDAYIRSFQQSWNEYAKRQGKSLADFASLCFHVPFTKMGKKALESIIDNADETTQERLRSGYEDAVDYNRYVGNIYTGSLYLSLISLLENRDLQAGETIGLFSYGSGSVGEFYSATLVEGYKDHLDQAAHKALLNNRTEVSVDAYETFFKRFDDVEFDEEQDAVHEDRHIFYLSNIENNVREYHRPE

SEQ ID NO: 3 - protein, 258 aa, Myxococcus xanthus
MPEFKVDARGPIEIWTIDGESRRNAISRAMLKELGELVTRVSSSRDVRAVVITGAGDKAFCAGADLKERATMAEDEVRAFLDGLRRTFRAIEKSDCVFIAAINGAALGGGTELALACDLRVAAPAAELGLTEVKLGIIPGGGGTQRLARLVGPGRAKDLILTARRINAAEAFSVGLANRLAPEGHLLAVAYGLAESVVENAPIAVATAKHAIDEGTGLELDDALALELRKYEEILKTEDRLEGLRAFAEKRAPVYKGR

SEQ ID NO: 4 - protein, 265 aa, Myxococcus xanthus
MKTARWCSLEEAVASIPDGASLATGGFMLGRAPMALVMELIAQGKRDLGLISLPNPLPAEFLVAGGCLARLEIAFGALSLQGRVRPMPCLKRAMEQGTLAWREHDGYRVVQRLRAASMGLPFIPAPDADVSGLARTEPPPTVEDPFTGLRVAVEPAFYPDVALLHARAADERGNLYMEDPTTDLLVAGAAKRVIATVEERVAKLPRATLPGFQVDRIVLAPGGALPTGCAGLYPHDDEMLARYLSLAETGREAEFLETLLTRRAA

SEQ ID NO: 5 - protein, 246 aa, Myxococcus xanthus
MSATLDITPAETVVSLLARQIDDGGVVATGVASPLAILAIAVARATHAPDLTYLACVGSLDPEIPTLLPSSEDLGYLDGRSAEITIPDLFDHARRGRVDTVFFGAAEVDAEGRTNMTASGSLDKPRTKFPGVAGAATLRQWVRRPVLLVPRQSRRNLVPEVQVATTRDPRRPVTLISDLGVFELGASGARLLARHPWASEEHIAERTGFAFQVSEALSVTSLPDARTVAAIRAIDPHGYRDALVGA

SEQ ID NO: 6 - protein, 132 aa, Escherichia coli
MSTTHNVPQGDLVLRTLAMPADTNANGDIFGGWLMSQMDIGGAILAKEIAHGRVVTVRVEGMTFLRPVAVGDVVCCYARCVQKGTTSVSINIEVWVKKVASEPIGQRYKATEALFKYVAVDPEGKPRALPVE

SEQ ID NO: 7 - protein, 477 aa, Lactobacillus brevis
MNTENIEQAIRKILSEELSNPQSSTATNTTVPGKNGIFKTVNEAIAATKAAQENYADQPISVRNKVIDAIREGFRPYIEDMAKRIHDETGMGTVSAKIAKLNNALYNTPGPEILQPEAETGDGGLVMYEYAPFGVIGAVGPSTNPSETVIANAIMMLAGGNTLFFGAHPGAKNITRWTIEKLNELVADATGLHNLVVSLETPSIESVQEVMQHPDVAMLSITGGPAVVHQALISGKKAVGAGAGNPPAMVDATANIALAAHNIVDSAAFDNNILCTAEKEVVVEAAVKDELIMRMQQEGAFLVTDSADIEKLAQMTIGPKGAPDRKFVGKDATYILDQAGISYTGTPTLIILEAAKDHPLVTTEMLMPILPVVCCPDFDSVLATATEVEGGLHHTASIHSENLPHINKAAHRLNTSIFVVNGPTYCGTGVATNGAHSGASALTIATPTGEGTATSKTYTRRRRLNSPEGFSLRTWEA

SEQ ID NO: 8 - protein, 1188 aa, Segniliparus rugosus
MTESQSYETRQARPAGQSLAERVARLVAIDPQAAAAVPDKAVAERATQQGLRLAQRIEAFLSGYGDRPALAQRAFEITKDPITGRAVATLLPKFETVSYRELLERSHAIASELANHAEAPVKAGEFIATIGFTSTDYTSLDIAGVLLGLTSVPLQTGATTDTLKAIAEETAPAVFGASVEHLDNAVTTALATPSVRRLLVFDYRQGVDEDREAVEAARSRLAEAGSAVLVDTLDEVIARGRALPRVALPPATDAGDDSLSLLIYTSGSTGTPKGAMYPERNVAQFWGGIWHNAFDDGDSAPDVPDIMVNFMPLSHVAGRIGLMGTLSSGGTTYFIAKSDLSTFFEDYSLARPTKLFFVPRICEMIYQHYQSELDRIGAADGSPQAEAIKTELREKLLGGRVLTAGSGSAPMSPELTAFIESVLQVHLVDGYGSTEAGPVWRDRKLVKPPVTEHKLIDVPELGYFSTDSPYPRGELAIKTQTILPGYYKRPETTAEVFDEDGFYLTGDVVAEVAPEEFVYVDRRKNVLKLSQGEFVALSKLEAAYGTSPLVRQISVYGSSQRSYLLAVVVPTPEALAKYGDGEAVKSALGDSLQKIAREEGLQSYEVPRDFIIETDPFTIENGILSDAGKTLRPKVKARYGERLEALYAQLAETQAGELRSIRVGAGERPVIETVQRAAAALLGASAAEVDPEAHFSDLGGDSLSALTYSNFLHEIFQVEVPVSVIVSAANNLRSVAAHIEKERSSGSDRPTFASVHGAGATTIRASDLKLEKFLDAQTLAAAPSLPRPASEVRTVLLTGSNGWLGRFLALAWLERLVPQGGKVVVIVRGKDDKAAKARLDSVFESGDPALLAHYEDLADKGLEVLAGDFSDADLGLRKADWDRLADEVDLIVHSGALVNHVLPYSQLFGPNVVGTAEVAKLALTKRLKPVTYLSTVAVAVGVEPSAFEEDGDIRDVSAVRSIDEGYANGYGNSKWAGEVLLREAYEHAGLPVRVFRSDMILAHRKYTGQLNVPDQFTRLILSLLATGIAPKSFYQLDATGGRQRAHYDGIPVDFTAEAITTLGLAGSDGYHSFDVFNPHHDGVGLDEFVDWLVEAGHPISRVDDYAEWLSRFETSLRGLPEAQRQHSVLPLLHAFAQPAPAIDGSPFQTKNFQSSVQEAKVGAEHDIPHLDKALIVKYAEDIKQLGLL

SEQ ID NO: 9 - protein, 225 aa, Segniliparus rugosus
MGLLNKVLATQVATAEAREDGPELVPYPGEEALIAQAAPKRRAEFITARRCAREALAKLKVPEQPILRGAKREPLWPKGVVGTLTHTAGFRGAAVAHSLVMRSVGADAEPHLPLPDGVLGQVALPEEQAWLAARGEDGLHWDRLLFCAKEATYKAWFPLTERWLGFEDARISFEESATDGADRTGSFHVQLLVPGQALKGPPLATFEGRWAVADGFIVTGISHGW

SEQ ID NO: 10 - protein, 316 aa, Escherichia coli
MSIKEQTLMTPYLQFDRNQWAALRDSVPMTLSEDEIARLKGINEDLSLEEVAEIYLPLSRLLNFYISSNLRRQAVLEQFLGTNGQRIPYIISIAGSVAVGKSTTAAVLQALLSRWPEHRRVELITTDGFLHPNQVLKERGLMKKKGFPESYDMHRLVKFVSDLKSGVPNVTAPVYSHLIYDVIPDGDKTVVQPDILILEGLNVLQSGMDYPHDPHHVFVSDFVDFSIYVDAPEDLLQTWYINRFLKFREGAFTDPDSYFHNYAKLTKEEAIKTAMTLWKEINWLNLKQNILPTRERASLILTKSANHAVEEVRLRK

SEQ ID NO: 11 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 12 - protein, 477 aa, Lactobacillus reuteri
MQINDIESAVRKILAEELDNASSSSANVTATTDNGHRGIFTNVNDAIAAAKAAQEIYRDKPIAVRQQVIDAIKEGFRPYIEKMAKDIKEETGMGTVEAKIAKLNNALYNTPGPEILEPVVENGDGGMVMYERLPYGVIGAVGPSTNPSETVIANAIMMLAGGNTLYFGAHPGAKNVTRWTIEKMNDFIADATGLHNLVVSIETPTIESVQQMMKHPDIAMLAVTGGPAVVHQAMTSGKKAVGAGPGNPPAMVDATADIDLAAHNIITSASFDNDILCTAEKEVVAESSIKDELIRKMQDEGAFVVNREQADKLADMCIQENGAPDRKFVGKDATYILDQANIPYTGHPVEIICELPKEHPLVMTEMLMPILPVVSCPTFDDVLKTAVEVEKGNHHTATIHSNNLKHINNAAHRMQCSIFVVNGPSYVGTGVADNGAHSGASALTIATPTGEGTCTARTFTRRVRLNSPQGFSVRNWY

SEQ ID NO: 13 - protein, 468 aa, Clostridium beijerinckii
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 14 - protein, 1151 aa, Mycobacterium kyorinense
MSIEINEERLERRIADLTATDQQFAAARPSQAVAAAVEQPGLTLPQVIQTVLEGYADRPALGQRAVEFVADPATGRTSAQLLPRFDTITYRELGERVGALASAWADGVVSPGDRVAVLGFTSVDYTTIDMALAQLGAVSVPLQTSAAIAQLQPIVAETEPTVLAASVDYLSDAVELIQSGRQPARLVVFDFHPEVDDHREALDAARARLANSPVAVETLADVLDRGKSLPPVPAVDTGNDALALLIYTSGSTGAPKGAMYPQRNVAKMWRRSARNWFGPSAASITLNFMPMSHVMGRGILYGTLGNGGTAYFGAKSDLSTLLEDLALVRPTELNFVPRIWETLYQEYQRELDRGRGEAEVLADMRQNLLGGRFIFAMTGSAPISPELNAWVESLLEMHLMDGYGSTEAGMVLFDGQVQRPPVIDYKLADVPDLGYFATDKPYPRGELLLKTENMFPGYYKRPEITADVLDADGYYHTGDVVAEVAPDRLVYVDRRNNVLKLAQGEFVTLAKLEAVFGTSPLVRQIYIYGNSAHPYLLAVVVPTDPNASKSAIAESLQTVAKEAGLQSYEVPRDFIIETTPFTLENGLLTGIRKLAWPKLKQHYGERLEQLYAELEESQANELRTLRQSGADKPVLETVSRAAAALLGASTSDVSPDAHFTDLGGDSLSALTFGNLLREIFDVDVPVGVIVSPASDLQSIAGYIEGERQGSKRPTFASVHGRDAVEVHAKDLTLDKFLDADTLAAAPNLPRANHEVRTVLLTGATGFLGRYLALEWLERMDLVDGKVIALVRAKDDAAARERLDKTFDSGEPKLLAHYQELAADHLEVIAGDKGEANLGLSQDVWQRLADTVDLIVDPAALVNHVLPYSELFGPNALGTAELIRIALTTKIKPYTYVSTIGVGDQIAPGKFTEDADVRVMSPTRAINDGYANGYGNSKWAGEVLLREANDLAGLPVAVFRCDMILADTTYAGQLNLPDMFTRMMLSLVATGIAPKSFYELDADGNRQRAHYDGLPVEFIAEAISTLGAQNVETFQTYHVMNPYDDGIGMDEYVDWLIEAGYPIQRIDDYREWVQRFETSLRALPEKQRQASLLPLLHNYQKPEKPIRGSIAPTDRFRAAVQEAKIGPDKDIPHVTAPVIVKYITDLQLLGLL

SEQ ID NO: 15 - protein, 1173 aa, Mycobacterium avium
MSTATHDERLDRRVHELIATDPQFAAAQPDPAITAALEQPGLRLPQIIRTVLDGYADRPALGQRVVEFVTDAKTGRTSAQLLPRFETITYSEVAQRVSALGRALSDDAVHPGDRVCVLGFNSVDYATIDMALGAIGAVSVPLQTSAAISSLQPIVAETEPTLIASSVNQLSDAVQLITGAEQAPTRLVVFDYHPQVDDQREAVQDAAARLSSTGVAVQTLAELLERGKDLPAVAEPPADEDSLALLIYTSGSTGAPKGAMYPQSNVGKMWRRGSKNWFGESAASITLNFMPMSHVMGRSILYGTLGNGGTAYFAARSDLSTLLEDLELVRPTELNFVPRIWETLYGEFQRQVERRLSEAGDAGERRAVEAEVLAEQRQYLLGGRFTFAMTGSAPISPELRNWVESLLEMHLMDGYGSTEAGMVLFDGEIQRPPVIDYKLVDVPDLGYFSTDRPHPRGELLLRTENMFPGYYKRAETTAGVFDEDGYYRTGDVFAEIAPDRLVYVDRRNNVLKLAQGEFVTLAKLEAVFGNSPLIRQIYVYGNSAQPYLLAVVVPTEEALASGDPETLKPKIADSLQQVAKEAGLQSYEVPRDFIIETTPFSLENGLLTGIRKLAWPKLKQHYGERLEQMYADLAAGQANELAELRRNGAQAPVLQTVSRAAGAMLGSAASDLSPDAHFTDLGGDSLSALTFGNLLREIFDVDVPVGVIVSPANDLAAIASYIEAERQGSKRPTFASVHGRDATVVRAADLTLDKFLDAETLAAAPNLPKPATEVRTVLLTGATGFLGRYLALEWLERMDMVDGKVIALVRARSNEEARARLDKTFDSGDPKLLAHYQQLAADHLEVIAGDKGEANLGLGQDVWQRLADTVDVIVDPAALVNHVLPYSELFGPNALGTAELIRLALTSKQKPYTYVSTIGVGDQIEPGKFVENADIRQMSATRAINDSYANGYGNSKWAGEVLLREAHDLCGLPVAVFRCDMILADTTYAGQLNLPDMFTRLMLSLVATGIAPGSFYELDADGNRQRAHYDGLPVEFIAAAISTLGSQITDSDTGFQTYHVMNPYDDGVGLDEYVDWLVDAGYSIERIADYSEWLRRFETSLRALPDRQRQYSLLPLLHNYRTPEKPINGSIAPTDVFRAAVQEAKIGPDKDIPHVSPPVIVKYITDLQLLGLL